(19) United States
(12) Patent Application Publication
(10) Pub. No.: US 2010/0152518 A1
(43) Pub. Date: Jun. 17, 2010

(54) PROCESS TO MAKE A LIQUID CATALYST HAVING A HIGH MOLAR RATIO OF ALUMINUM TO NITROGEN

(75) Inventors: Howard S. Lacheen, Richmond, CA (US); Saleh Elomari, Fairfield, CA (US)

Correspondence Address: CHEVRON CORPORATION, P.O. BOX 6006, SAN RAMON, CA, 94583-0806, US

(73) Assignee: Chevron U.S.A., Inc.

(21) Appl. No.: 12/335,494

(22) Filed: Dec. 15, 2008

(51) Int. Cl. C07C 2/58; B01J 31/26

(52) U.S. Cl. 585/722; 502/159

(57) ABSTRACT

A process to make a liquid catalyst having a molar ratio of Al to N greater than 2.0, comprising: a) using an ammonium-based ionic liquid catalyst to catalyze a reaction, wherein the ammonium-based ionic liquid catalyst builds up an impurity during the reaction; and b) mixing the ammonium-based ionic liquid catalyst, having an impurity, with aluminum. There is also provided a process for isoparaffin/olefin alkylation, wherein the ionic liquid catalyst comprises a quaternary ammonium ionic liquid salt; and wherein the ionic liquid catalyst has a molar ratio of Al to N greater than 2.0 when held at a temperature at or below 25° C. for at least two hours. There is also provided a method for making a catalyst having a molar ratio of Al to N greater than 2.0, and a process for hydroconversion comprising maintaining a level of conjunct polymer in an ionic liquid catalyst.

This application is related to co-filed patent applications titled “An Ionic Liquid Catalyst Having a High Molar Ratio of Aluminum to Nitrogen” and “A Process for Hydrocarbon Conversion Using, A Method to Make, and Compositions of, an Acid Catalyst,” herein incorporated by reference in their entireties.

### FIELD OF THE INVENTION

This invention is directed towards a process to make a liquid catalyst, a process for isoparaffin/olefin alkylation, a method to make an ionic liquid catalyst, and a process for hydrocarbon conversion.

### DETAILED DESCRIPTION OF THE INVENTION

A process to make a liquid catalyst having a molar ratio of Al to N greater than 2.0 is provided. The process comprises:
a. using an ammonium-based ionic liquid catalyst to catalyze a reaction, wherein the ammonium-based ionic liquid catalyst builds up an impurity during the reaction; and
b. mixing the ammonium-based ionic liquid catalyst, having an impurity, with aluminum to make a liquid catalyst having a molar ratio of Al to N greater than 2.0, wherein the liquid catalyst having a molar ratio of Al to N greater than 2.0, is effective for catalyzing the reaction.

There is also provided a process for alkylation, comprising: contacting an ionic liquid catalyst with an olefin and an isoparaffin; wherein the olefin and the isoparaffin are alkylated; wherein the ionic liquid catalyst comprises a quaternary ammonium ionic liquid salt; and wherein the ionic liquid catalyst has a molar ratio of Al to N greater than 2.0 when held at a temperature at or below 25° C. for at least two hours.

In a separate embodiment, there is provided a method to make a catalyst. The method comprises mixing an ionic liquid catalyst comprising an impurity, with aluminum chloride. The mixing step creates a mixed ionic liquid catalyst that has a molar ratio of Al to N greater than 2.0. The mixed ionic liquid catalyst is effective for catalyzing a reaction.

There is also provided a process for hydrocarbon conversion, comprising:
a. using an ionic liquid catalyst for hydrocarbon conversion, whereby a conjunct polymer builds up in the ionic liquid catalyst;
b. adding aluminum to the ionic liquid catalyst; and
c. maintaining a level of the conjunct polymer in the ionic liquid catalyst in a range such that the ionic liquid catalyst may be used for an extended period.

In different embodiments the impurity comprises, consists of, or consists essentially of conjunct polymers.

Definitions:

The term “comprising” means including the elements or steps that are identified following that term, but any such elements or steps are not exhaustive, and an embodiment may include other elements or steps.

“Ionic liquids” are liquids whose make-up is comprised of ions as a combination of cations and anions. The most common ionic liquids are those prepared from organic-based cations and inorganic or organic anions. Ionic liquid catalysts are used in a wide variety of reactions, including Friedel-Crafts reactions.

“Alkyl” means a linear saturated hydrocarbon of one to nine carbon atoms or a branched saturated hydrocarbon of three to twelve carbon atoms. In one embodiment, the alkyl groups are methyl. Examples of alkyl groups include, but are not limited to, groups such as methyl, ethyl, n-propyl, isopropyl, n-butyl, isobutyl, sec-butyl, t-butyl, n-pentyl, and the like.

“Effective hydrocarbon conversion” means that a commercially sufficient amount of the hydrocarbon is converted. For example, in an isoparaffin/olefin alkylation this could be greater than 75 wt % conversion of an olefin, greater than 85 wt % conversion of an olefin, greater than 95 wt % conversion of an olefin, or up to 100 wt % conversion of an olefin. The commercially significant amount can vary substantially depending on the hydrocarbon being converted and the value of the converted product that is produced.

### Ionic Liquid Catalyst:

The ionic liquid catalyst is composed of at least two components which form a complex. To be effective at alkylation the ionic liquid catalyst is acidic. The ionic liquid catalyst comprises a first component and a second component. The first component of the catalyst will typically comprise a strong Lewis acid. Lewis acids that are useful for alkylations include, but are not limited to, aluminum halides, gallium halides, indium halides, iron halides, tin halides and titanium halides. In one embodiment the first component is aluminum halide. For example, aluminum trichloride (AlCl₃) may be used as the first component for preparing the ionic liquid catalyst.

The second component making up the ionic liquid catalyst is an organic salt or mixture of salts. These salts may be characterized by the general formula Q⁺A⁻, wherein Q⁺ is an ammonium, phosphonium, or sulfonium cation and A⁻ is a negatively charged ion such as Cl⁻, Br⁻, ClO₄⁻, NO₃⁻, BF₄⁻, BCl₄⁻, PF₆⁻, SbF₆⁻, AlCl₄⁻, Al₂Cl₇⁻, Al₃Cl₁₀⁻, AlF₆⁻, TaF₆⁻, CuCl₂⁻, FeCl₃⁻, SO₃CF₃—, and 3-sulfurtrioxyphenyl. In one embodiment the second component is selected from those having quaternary ammonium halides containing one or more alkyl moieties having from about 1 to about 9 carbon atoms, such as, for example, trimethylammonium hydrochloride, methyltributylammonium, 1-butylpyridinium, or alkyl substituted imidazolium halides, such as for example, 1-ethyl-3-methyl-imidazolium chloride.

In one embodiment the Al is in the form of AlCl₃ and the N is in the form of R₄N⁺X⁻ or R₃NH⁺X⁻, where R is an alkyl group and X is a halide. Examples of halides that can be used are chloride, bromide and iodide.

In one embodiment the ionic liquid catalyst is a quaternary ammonium chloroaluminate ionic liquid having the general formula RR′R″NH⁺Al₂Cl₇⁻, wherein RR′ and R″ are alkyl groups containing 1 to 12 carbons. Examples of quaternary ammonium chloroaluminate ionic liquid salts are N-alkyl-pyridinium chloroaluminate, N-alkyl-alkylpyridinium chloroaluminate, pyridinium hydrogen chloroaluminate, alkylpyridinium hydrogen chloroaluminate, di-alkyl-imidazolium chloroaluminate, tetra-alkyl-ammonium chloroaluminate, tri-alkyl-ammonium hydrogen chloroaluminate, or a mixture thereof.

The presence of the first component should give the ionic liquid a Lewis or Franklin acidic character. Generally, the greater the mole ratio of the first component to the second component, the greater is the acidity of the ionic liquid mixture.

For example, a typical reaction mixture to prepare n-butyl pyridinium chloroaluminate ionic liquid salt having a molar ratio of Al to N of no greater than 2.0 is shown below:

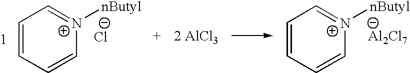

For the case of the above reaction, and for typical quaternary ammonium chloroaluminate salts, the molar ratio of Al to N cannot exceed 2.0 at room temperature for extended periods. This is because any additional AlCl₃ precipitates out and would not stay in the ionic liquid.

The molar ratio of Al to N in the ionic liquid catalyst is higher than what is possible in a freshly prepared quaternary ammonium chloroaluminate salt or alkyl pyridinium haloaluminate ionic liquid, which have a maximum molar ratio of Al to N of 2.0. In some embodiments the molar ratio of Al to N is greater than 2.1, greater than 2.5, or even greater than 2.8. In some embodiments the molar ratio of Al to N is less than 9, less than 8, less than 5, or less than 4. In one embodiment the molar ratio of Al to N is from 2.1 to 8; such as, for example, from 2.5 to 5.1 or from 2.5 to 4.

In some embodiments the molar ratio, or the level of the impurity, are controlled to remain in a suitable range for effective hydrocarbon conversion. The molar ratio, or the level of the impurity, can be maintained for example by adjusting the rate of addition of aluminum, maintaining a level of conjunct polymer in the ionic liquid catalyst, or by adjusting the level of a halide or a Broensted acid, performing partial ionic liquid catalyst regeneration on a slip-stream, or combinations thereof. In one embodiment the process to make the liquid catalyst comprises maintaining a level of the impurity between 1 and 24 wt %.

In one aspect, the ionic liquid catalyst comprises an impurity in the catalyst that increases the catalyst's capacity to uptake AlCl₃. In one embodiment the catalyst comprises one or more, conjunct polymers as an impurity which increases the catalyst's capacity to uptake AlCl₃. In this embodiment the level of the conjunct polymer is present in an amount that still enables the ionic liquid catalyst or catalyst system to perform its desired catalytic function.

The level of the impurity (e.g., conjunct polymer) will generally be less than or equal to 30 wt %, but examples of other desired rarnges of impurity in the ionic liquid catalyst or catalyst system are from 1 to 24 wt %, from 1 to 20 wt %, from 0.5 to 15 wt %,: or from 0.5 to 12 wt %.

The term conjunct polymer was first used by Pines and Ipatieff to distinguish these polymeric molecules from typical polymers. Unlike typical polymers which are compounds formed from repeating units of smaller molecules by controlled or semi-controlled polymerizations, “conjunct polymers” are “pseudo-polymeric” compounds formed asymmetrically from two or more reacting units by concurrent acid-catalyzed transformations including polymerization, alkylation, cyclization, additions, eliminations and hydride transfer reactions. Consequently, the produced “pseudo-polymeric” may include a large number of compounds with varying structures and substitution patterns. The skeletal structures of “conjunct polymers”, therefore, range from the very simple linear molecules to very complex multi-feature molecules.

Some examples of the likely polymeric species in conjunct polymers were reported by Miron et al. (Journal of Chemical and Engineering Data, 1963), and Pines (Chem. Tech, 1982). Conjunct polymers are also commonly known to those in the refining industry as “red oils” due to their reddish-amber color or “acid-soluble oils” due to their high uptake in the catalyst phase where paraffinic products and hydrocarbons with low olefinicity and low functional groups are usually immiscible in the catalyst phase. In this application, the term “conjunct polymers” also includes ASOs (acid-soluble-oils) and red oils.

The level of conjunct polymer in the acid catalyst is determined by hydrolysis of known weights of the catalyst. An example of a suitable test method is described in Example 3 of commonly assigned U.S. Patent Publication Number US20070142213A1. Conjunct polymers can be recovered from the acid catalyst by means of hydrolysis. The hydrolysis recovery methods employ procedures that lead to complete recovery of the conjunct polymers and are generally used for analytical and characterization purposes because it results in the destruction of the catalyst. Hydrolysis of the acid catalyst is done, for example, by stirring the spent, catalyst in the presence of excess amount of water followed by extraction with low boiling hydrocarbon solvents such as pentane or hexane. In the hydrolysis process, the catalyst salt and other salts formed during hydrolysis go into the aqueous layer while conjunct polymers go into the organic solvent. The low boiling solvent containing the conjunct polymers are concentrated on a rotary evaporator under vacuum and moderate temperature to remove the extractant, leaving behind the high boiling residual oils (conjunct polymers) which are collected and analyzed. The low boiling extractants can be also removed by distillation methods.

In one embodiment the ionic liquid catalyst comprises greater than 1 wt % conjunct polymer. In one embodiment, the higher the level of conjunct polymer in the ionic liquid catalyst or catalyst system the higher is the molar ratio of Al to N. This is because the catalysts capacity for uptake of AlCl₃ increases at higher conjunct polymer concentration in the catalyst phase.

In one embodiment, the solubility of incremental AlCl₃ above the 2.0 Al/N molar ratio in the ionic liquid catalyst or catalyst system is 3 wt % or higher at 50° C. or below. In other embodiments the solubility of incremental AlCl₃ above the 2.0 Al/N molar ratio in the ionic liquid catalyst or catalyst system is from 3 wt % to 20 wt %, or from 4 wt % to 15 wt % at 50° C. or below.

In one embodiment, the solubility of incremental AlCl₃ above the 2.0 Al/N molar ratio in the ionic liquid catalyst or catalyst system is significantly higher at 100° C. than at 50° C. For example the solubility of incremental AlCl₃ above the 2.0 Al/N molar ratio in the ionic liquid catalyst or catalyst system can be greater than 10 wt % at 100° C., such as from 12 to 50 wt %, from 12 to 40 wt %, or from 15 to 35 wt % at 100° C. In one embodiment the solubility of incremental AlCl₃ above the 2.0 Al/N molar ratio in the ionic liquid catalyst or catalyst system is at least 10 wt % higher at 100° C. than at 50° C.

In one embodiment, the AlCl₃ that is soluble and stable in the ionic liquid catalyst or catalyst system remains soluble in the ionic liquid catalyst or catalyst system. An example of this is where less than 0.1 wt %, less than 0.05 wt %, less than 0.01 wt %, or zero wt % AlCl₃ precipitates out of the ionic liquid catalyst or catalyst system when it is held for three hours or longer at 25° C. or below.

In one embodiment the conjunct polymer is extractable. The conjunct polymer may be extracted during a catalyst regeneration process, such as by treatment of the catalyst with aluminum metal or with aluminum metal and hydrogen chloride. Examples of methods for regenerating ionic liquid catalysts are taught in U.S. Patent Publications US20070142215A1, US20070142213A1, US20070142676A1, US20070142214A1, US20070142216A1, US20070142211A1, US20070142217A1, US20070142218A1, US20070249485 A1, and in U.S. patent application Ser. No. 11/960,319, filed Dec. 19, 2007; Ser. No. 12/003,577, filed Dec. 28, 2007; Ser. No. 12/003,578, filed Dec. 28, 2007; Ser. No. 12/099,486, filed Apr. 8, 2008; and 61/118,215, filed Nov. 26, 2008.

The mixing of aluminum with the ammonium-based ionic liquid catalyst can be done in a continuous reactor process, for example by taking a portion or the entire volume of the effluent from an alkylation reactor and mixing it with aluminum before it is recycled back to the alkylation reactor. The ammonium-based ionic liquid catalyst can be used continuously without having to be removed from the continuous reactor process for more than 7 days, more than 25 days, or more than 50 days.

In some embodiments the ionic liquid catalyst is useful for catalyzing a hydrocarbon conversion reaction. One example of a hydrocarbon conversion reaction is a Friedel-Crafts reaction. Other examples are alkylation, isomerization, hydrocracking, polymerization, dimerization, oligomerization, acylation, acetylation, metathesis, copolymerization, hydroformylation, dehalogenation, dehydration, olefin hydrogenation and combinations thereof. For example, some of the ionic liquid catalysts are used for isoparaffin/olefin alkylation. Examples of ionic liquid catalysts and their use for isoparaffin/olefin alkylation are taught, for example, in U.S. Pat. Nos. 7,432,408 and 7,432,409, 7,285,698, and U.S. patent application Ser. No. 12/184,069, filed Jul. 31, 2008. A high quality gasoline blending component, a middle distillate, or a mixture thereof can be made from these processes. In some embodiments the alkylate from the isoparaffin/olefin alkylation has a Research-method octane number (RON) of 86 or higher, or even 92 or higher. The RON is determined using ASTM D 2699-07a. Additionally, the RON may be calculated [RON (GC)] from gas chromatography boiling range distribution data.

In some embodiments very little or no solids precipitate out of the ionic liquid catalyst when it is held at a temperature at or below 25° C. for an extended time. The time the catalyst is held at a temperature at or below 25° C. can be fairly lengthy. In general, the time is for greater than a minute, but it can be much longer, such as for greater than 5 minutes, for at least two hours, three hours or longer, up to two weeks, more than 50 days, several months, or even a year.

In some embodiments, the mixing with aluminum is done in the presence of a Broensted acid, such as a hydrogen halide; for example, hydrogen chloride. In other embodiments, the mixing with aluminum is done in the absence of a Broensted acid. In some embodiments, the ionic liquid catalyst additionally comprises a Broensted acid. In one embodiment, the hydrogen halide is at least partially produced from an alkyl halide. In one embodiment, the hydrogen halide increases the acidity, and thus the activity of the ionic liquid catalyst. In one embodiment, the hydrogen halide, in combination with aluminum, assists in the conversion of the inactive anion, e.g., AlCl₄⁻to form the more acidic and effective chloroaluminate species for alkylation, such as AlCl₃, Al₂Cl₇⁻, or even Al₃Cl₁₀⁻. In some embodiments, the alkyl halide is derived from the isoparaffin or olefin used in a given reaction. For example, with the alkylation of isobutene with butane in chloroaluminate ionic liquids, the alkyl halide could be 1-butyl chloride, 2-butyl chloride, t-butyl chloride, or a mixture thereof. Other examples of alkyl halides that can be used are ethyl chloride, isopentyl chloride, hexyl chloride, or heptyl chloride. In one embodiment, the amount of the alkyl chloride should be kept at low concentrations and not exceed the molar concentration of the Lewis acid portion of the catalyst, AlCl₃. In one embodiment, the amounts of the alkyl chloride used may range from 0.05 mol % to 100 mol % of the Lewis acid portion of the ionic liquid catalyst, AlCl₃. The amount of the alkyl chloride can be adjusted to keep the acidity of the ionic liquid catalyst or ionic liquid catalyst system at the desired performing capacity. In another embodiment, the amount of the alkyl chloride is proportional to the olefin, and does not exceed the molar concentration of the olefin in the isoparaffin/olefin alkylation reaction.

Any term, abbreviation or shorthand not defined is understood to have the ordinary meaning used by a person skilled in the art at the time the application is filed. The singular forms “a,” “an,” and “the,” include plural references unless expressly and unequivocally limited to one instance.

All of the publications, patents and patent applications cited in this application are herein incorporated by reference in their entirety to the same extent as if the disclosure of each individual publication, patent application or patent was specifically and individually indicated to be incorporated by reference in its entirety.

This written description uses examples to disclose the invention, including the best mode, add also to enable any person skilled in the art to make and use the invention. Many modifications of the exemplary embodiments of the invention disclosed above will readily occur to those skilled in the art. Accordingly, the invention is to be construed as including all structure and methods that fall within the scope of the appended claims.

### EXAMPLES

### Example 1

An isobutane-butene alkylation catalyzed with butyl pyridinium chloroaluminate ionic liquid, and co-catalyzed with t-butyl chloride, was performed in a continuous liquid phase reactor. During the alkylation, the ionic liquid catalyst was continuously regenerated by mixing it with aluminum metal at 100° C. after each pass through the alkylation reactor. The aluminum metal regeneration treatment reactivated the catalyst by removing most of the conjunct polymers that accumulated as alkylation by-products in the catalyst phase and by making and re-making AlCl₃. The regeneration resulted in the formation of excess AlCl₃, depending on how much chloride sank into the catalyst phase from the alkyl chloride used as a co-catalyst.

The level of conjunct polymer in the ionic liquid catalyst was maintained between 2 and 23 wt % during the alkylation. Elemental analysis of the ionic liquid showed that the molar ratio of Al to N increased over time during the alkylation with no precipitation of excess AlCl₃ formed during the continuous generation cycles. The freshly prepared ionic liquid, with no conjunct polymer, had a molar ratio of Al to N of 2.0. During alkylation, the molar ratio of Al to N in the liquid catalyst increased to 2.1, and then to 2.5 and then to 4.0 when sampled over a period of greater than 50 days. The molar ratio of Al to N in the liquid catalyst was maintained between 2.1 and 8.0. Even with a higher molar ratio of Al to N, the ionic liquid catalyst still remained effective for alkylation and produced an alkylate product with a RON greater than 92. The higher molar ratio of Al to N in the catalyst with conjunct polymer extended the life of the ionic liquid catalyst before it required complete regeneration.

### Example 2

The solubility of incremental AlCl₃ above the 2.0 Al/N molar ratio in the different samples of n-butyl pyridinium chloroaluminate ionic liquid catalyst with different levels of conjunct-polymer impurity were tested at four different temperatures. The solubility study results are summarized in Table 1, below.

TABLE 1

| | Incremental AlCl₃ Solubility, | | | |
|---|---|---|---|---|
| | Wt % | | | |
| | 25° C. | 50° C. | 75° C. | 100° C. |
| Fresh Catalyst with 0 wt % | 1.6 | 2 | 6.3 | 9.8 |
| Conjunct Polymer | | | | |
| Regenerated Catalyst with ~2 wt % | 4 | 8 | 22 | 26 |
| Conjunct Polymer | | | | |
| Regenerated Catalyst with 11 wt % | 8.4 | 9 | 22 | 29 |
| Conjunct Polymer | | | | |
| Spent Catalyst with15 wt % | 9 | 10 | 24 | 33 |
| Conjunct Polymer | | | | |

All of the samples of catalyst comprising conjunct polymer had a solubility of incremental AlCl₃ in the ionic liquid catalyst that was at least 10 wt % higher at 100° C. than at 50° C. The samples, with various amounts of solubilized incremental AlCl₃, were moved to room temperature and observed over time for AlCl₃ precipitation. Room temperature was approximately 25° C. or below.

All of the incremental AlCl₃ that was initially soluble in the fresh catalyst precipitated out within two hours of standing at room temperature. Approximately 75% of the incremental AlCl₃ that was originally soluble in the regenerated catalyst with ˜2 wt % conjunct polymer precipitated out within 72 hours of standing at room temperature;

A slight amount of incremental AlCl₃ precipitated out of the regenerated catalyst with 11 wt % conjunct polymer when it was held at room temperature overnight. No substantial additional amount precipitated out over a two week period of standing at room temperature.

No precipitation was observed in the spent catalyst samples held at room temperature for over two weeks.

What is claimed is:

1. A process to make a liquid catalyst having a molar ratio of Al to N greater than 2.0, comprising:
a. using an ammonium-based ionic liquid catalyst to catalyze a reaction, wherein the ammonium-based ionic liquid catalyst builds up an impurity during the reaction; and
b. mixing the ammonium-based ionic liquid catalyst, having an impurity, with aluminum to make a liquid catalyst having a molar ratio of Al to N greater to than 2.0, wherein the liquid catalyst having a molar ratio of Al to N greater than 2.0 is effective for catalyzing the reaction.

2. The process of claim 1, wherein the mixing is done at conditions to produce soluble aluminum chloride.

3. The process of claim 1, wherein the mixing with aluminum is done in the presence of a Broensted acid.

4. The process of claim 3, wherein the Broensted acid is hydrogen chloride.

5. The process of claim 1, wherein the mixing with aluminum is done in the absence of a Broensted acid.

6. The process of claim 1, additionally comprising maintaining a level of the impurity between 1 and 24 wt %.

7. The process of claim 6, wherein the level is between 2 and 23 wt %.

8. The process of claim 1, wherein the reaction is a hydrocarbon conversion selected from the group of alkylation, isomerization, hydrocracking, polymerization, dimerization, oligomerization, acylation, acetylation, metathesis, copolymerization, hydroformylation, dehalogenation, dehydration, olefin hydrogenation, or combinations thereof.

9. The process of claim 8, wherein the reaction is an alkylation.

10. The process of claim 9, wherein the reaction is an isoparaffin/olefin alkylation.

11. The process of claim 1, wherein the impurity comprises one or more conjunct polymers.

12. The process of claim 1, wherein the liquid catalyst having a molar ratio of Al to N greater than 2.0 comprises from 1 to 24 wt % of the impurity.

13. The process of claim 1, wherein the ammonium-based ionic liquid catalyst is a quaternary ammonium chloroaluminate ionic liquid salt.

14. The process of claim 13, wherein the quaternary ammonium chloroaluminate ionic liquid salt is selected from the group consisting of an N-alkyl-pyridinium chloroaluminate, a N-alkyl-alkylpyridinium chloroaluminate, a pyridinium hydrogen chloroaluminate, an alkylpyridinium hydrogen chloroaluminate, a di-alkyl-imidazolium chloroaluminate, a tetra-alkyl-ammonium chloroaluminate, a tri-alkyl-ammonium hydrogen chloroaluminate, or a mixture thereof.

15. The process of claim 1, wherein the molar ratio of the Al to N is from 2.1 to 8.0.

16. The process of claim 1, wherein the Al is in the form of AlCl₃ and the N is in the form of R₄N⁺X⁻ or R₃NH⁺X⁻, where R is an alkyl group and X is a halide.

17. The process of claim 1, wherein, after mixing, less than 0.1 wt % AlCl₃ precipitates out of the liquid catalyst having a molar ratio of Al to N greater than 2.0, when it is held for three hours or longer at 25° C. or below.

18. The process of claim 1, wherein the mixing is done in a continuous reactor process.

19. A process for alkylation, comprising: contacting an ionic liquid catalyst with an olefin and an isoparaffin; wherein the olefin and the isoparaffin are alkylated; wherein the ionic liquid catalyst comprises a quaternary ammonium ionic liquid salt; and wherein the ionic liquid catalyst has a molar ratio of Al to N greater than 2.0, when held at a temperature at or below 25° C. for at least two hours.

20. The, process of claim 19, wherein the contacting step produces an alkylate selected from the group of a gasoline blending component, a middle distillate, or a mixture thereof.

21. The process of claim 19, wherein the gasoline blending component has a RON greater than 86.

22. The process of claim 19, wherein the ionic liquid catalyst additionally comprises greater than 1 wt % conjunct polymer.

23. The process of claim 19, wherein the ionic liquid catalyst additionally comprises a Broensted acid.

24. The process of claim 23, wherein the Broensted acid is a hydrogen halide.

25. The process of claim 24, wherein the hydrogen halide is at least partially produced from an alkyl halide.

26. The process of claim 19, wherein the molar ratio of Al to N is from 2.1 to 8.0.

27. The process of claim 19, wherein the ionic liquid catalyst has a solubility of incremental AlCl₃ above the 2.0 Al/N molar ratio in the ionic liquid catalyst from 3 to 100 wt % at 100° C. or below.

28. The process of claim 19, wherein the solubility of incremental AlCl₃ in the ionic liquid catalyst is at least 10 wt % higher at 100° C. than at 50° C.

29. The process of claim 19, wherein, after mixing, less than 0.1 wt % AlCl₃ precipitates out of the ionic liquid catalyst, when it is held for three hours or longer at 25° C. or below.

30. The process of claim 19, additionally comprising maintaining a level of an impurity in the ionic liquid catalyst between 1 and 24 wt %.

31. A method to make a catalyst, comprising: mixing an ionic liquid catalyst comprising an impurity, with aluminum chloride to make a mixed ionic liquid catalyst; whereby the mixed ionic liquid catalyst has a molar ratio of Al to N greater than 2.0; and wherein the mixed ionic liquid catalyst is effective for catalyzing a reaction.

32. The method of claim 31, wherein the mixing is done in the presence of a Bronstead acid.

33. The method of claim 31, wherein the impurity comprises one or more conjunct polymers.

34. The method of claim 31, wherein the level of the impurity is between 1 to 24 wt %

35. The method of claim 31, wherein the mixing is done in a continuous reactor process.

36. The method of claim 31, wherein the mixing is done on an effluent from an alkylation reactor.

37. The method of claim 31, wherein the reaction is a hydrocarbon conversion reaction selected from the group of alkylation, isomerization, hydrocracking, polymerization, dimerization, oligomerization, acylation, acetylation, metathesis, copolymerization, hydroformylation, dehalogenation, dehydration, olefin hydrogenation, or combinations thereof.

38. The method of claim 31, wherein the ionic liquid catalyst comprising an impurity is selected from the group consisting of N-alkyl-pyridinium chloroaluminate, N-alkyl-alkylpyridinium chloroaluminate, a pyridinium hydrogen chloroaluminate, an alkylpyridinium hydrogen chloroaluminate, a di-alkyl-imidazolium chloroaluminate, a tetra-alkyl-ammonium chloroaluminate, a tri-alkyl ammonium hydrogen chloroaluminate, and mixtures thereof.

39. The method of claim 31, wherein the molar ratio is from 2.1 to 8.0.

40. The method of claim 31, wherein, after mixing, less than 0.1 wt % AlCl₃ precipitates out of the liquid catalyst having a molar ratio of Al to N greater than 2.0, when it is held for three hours or longer at 25° C. or below.

41. A process for hydrocarbon conversion, comprising:
a. using an ionic liquid catalyst for a hydrocarbon conversion whereby a conjunct polymer builds up in the ionic liquid catalyst;
b. adding aluminum to the ionic liquid catalyst; and
c. maintaining a level of the conjunct polymer in the ionic liquid catalyst in a range such that the ionic liquid catalyst may be used for an extended period for the hydrocarbon conversion.
